(19) United States
(12) Patent Application Publication
(10) Pub. No.: US 2014/0135603 A1
(43) Pub. Date: May 15, 2014

(54) SYSTEMS AND METHODS FOR PATIENT MONITORING

(71) Applicant: COVIDIEN LP, Mansfield, MA (US)

(72) Inventors: Robert T. Boyer, Longmont, CO (US); William A. Jordan, Westminster, CO (US)

(73) Assignee: COVIDIEN LP, Mansfield, MA (US)

(21) Appl. No.: 13/674,701

(22) Filed: Nov. 12, 2012

(51) Int. Cl. A61B 5/00; A61B 5/0205; A61B 5/1455

(52) U.S. Cl. CPC A61B 5/7275; A61B 5/14551; A61B 5/0205; A61B 5/0004; A61B 5/742
USPC 600/324

(57) ABSTRACT

Systems and methods for patient monitoring that include receiving a set first of SpO₂ readings from a first sensor over a time period, receiving a set of second SpO₂ readings from a second sensor over the time period, calculating an average first SpO₂ value based on some or all of the first SpO₂ readings, calculating an average second SpO₂ value based on some or all of the second SpO₂ readings, calculating a differential value based some or all of the first SpO₂ readings and some or all of the second SpO₂ readings, and determining a screen result based upon the average first SpO₂ value, the average second SpO₂ value, and the differential value. The differential value may be, for example, an average differential value or an accumulation differential value.

### BACKGROUND

1. Technical Field

The present disclosure relates to patient monitoring and, more particularly, to systems and methods for monitoring the oxygen saturation of a patient's blood.

2. Background of Related Art

In the United States, about 4,800 babies are born every year with critical congenital heart defects (“CCHDs”), which pose significant risks to babies whose conditions go undiagnosed. In an effort to diagnose CCHDs at an early stage, screening for CCHDs has been added to the Recommended Uniform Screening Panel for newborns.

Pulse oximetry screening is a non-invasive technique used to measure the percent oxygen saturation of hemoglobin in a patient's arterial blood (SpO₂). Pulse oximetry screening is used as a diagnostic tool for diagnosing CCHDs in newborns, as low SpO₂ levels may potentially indicate the presence of a CCHD. However, pulse oximetry screening is also used to monitor SpO₂ levels in other applications, e.g., to monitor SpO₂ levels above and below a surgical site after an arterial-related surgery.

### SUMMARY

The present disclosure relates to systems and methods for screening a patient, generally including receiving a set of first SpO₂ readings from a first sensor over a time period, receiving a set of second SpO₂ readings from a second sensor over the time period, calculating an average first SpO₂ value based on some or all of the first SpO₂ readings, calculating an average second SpO₂ value based on some or all of the second SpO₂ readings, calculating a differential value based on some or all of the first SpO₂ readings and some or all of the second SpO₂ readings, and determining a screen result based upon the average first SpO₂ value, the average second SpO₂ value, and the differential value. The differential value may be, for example, an average differential value or an accumulation differential value.

The aspects and features of the present disclosure are advantageous in that they provide for a more accurate indication of the SpO₂ levels in a patient's blood, reducing the likelihood of false results (false negatives and false positives) that may occur due to unreliable data and/or data abnormalities, e.g., spikes, valleys, etc., at any particular point-in-time. The aspects and features of the present disclosure are also advantageous in that they allow for the exclusion of data from a particular period of time (or particular periods of time) determined to be unreliable, thus reducing the influence of unreliable data on the overall results. The aspects and features of the present disclosure are further advantageous in that they provide for the display of current and average data as well as additional SpO₂ level metrics on a bedside device and/or a remote device, thus allowing a user, e.g., a healthcare provider, to readily ascertain both the current status of the patient and the status of the patient over an elapsed period of time.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic illustration of a patient monitoring system provided in accordance with the present disclosure;

FIG. 2 is a schematic illustration of one hardware and software configuration for use with the patient monitoring system of FIG. 1;

FIG. 3 illustrates an exemplary main display screen as displayed by a user interface or other display associated with the patient monitoring system of FIG. 1;

FIG. 4 illustrates an exemplary setting screen as displayed by a user interface or other display associated with the patient monitoring system of FIG. 1;

FIG. 5 is a flow diagram illustrating a method of patient monitoring provided in accordance with the present disclosure;

FIG. 6 is a flow diagram of the method of patient monitoring illustrated in FIG. 5 with the “SCREEN” step provided in greater detail;

FIG. 7 is a flow diagram of the method of patient monitoring illustrated in FIG. 5 with the “ANALYZE SCREEN RESULTS” step provided in greater detail;

FIG. 8 is a flow diagram of the method of patient monitoring illustrated in FIG. 5 with another embodiment of the “ANALYZE SCREEN RESULTS” step provided in greater detail;

FIG. 9 is a flow diagram of the method of patient monitoring illustrated in FIG. 5 with another embodiment of the “ANALYZE SCREEN RESULTS” step provided in greater detail; and

FIG. 10 is a flow diagram of the method of patient monitoring illustrated in FIG. 5 with the “DETERMINE SCREEN RESULT” step provided in greater detail.

### DETAILED DESCRIPTION

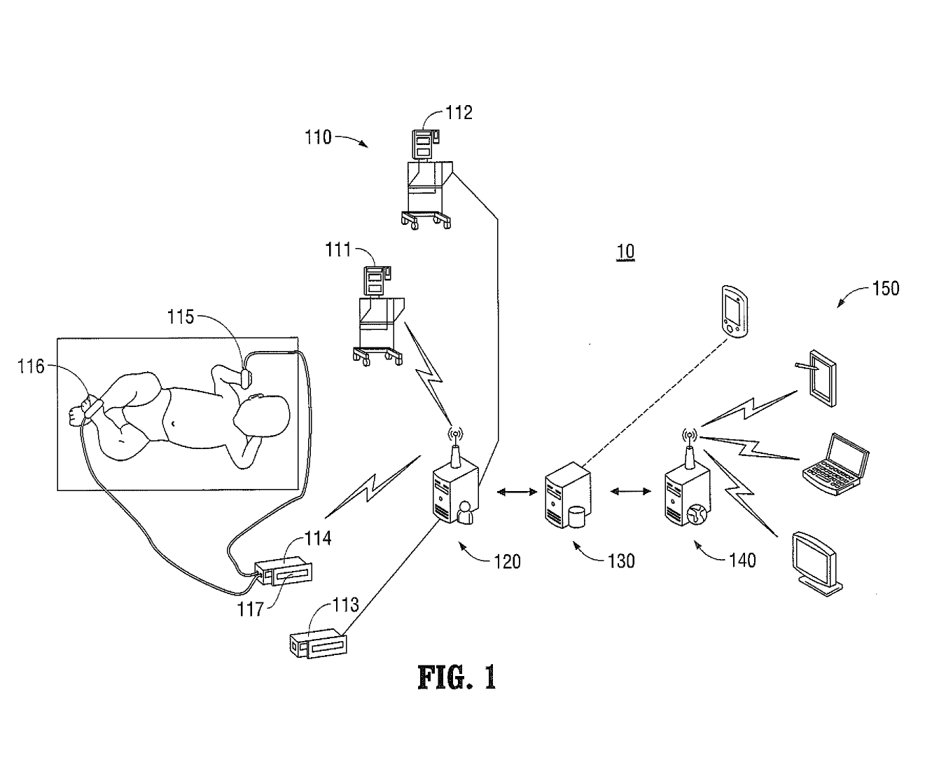

Referring to FIG. 1, an exemplary patient monitoring system provided in accordance with the present disclosure is shown generally identified by reference numeral 10. System 10 includes one or more patient monitoring devices 110, a data server 120, an application server 130, a web server 140, and one or more remote devices 150. For the purposes herein, exemplary patient monitoring system 10 is generally described, although the aspects and features of the present disclosure may be implemented, incorporated, or utilized with any suitable patient monitoring devices or systems.

Patient monitoring devices 110 may include one or more bedside monitoring devices 111, 112 one or more portable monitoring devices 113, 114, and/or any other suitable device(s) for visual monitoring, audible monitoring, monitoring of physical characteristics, physiological conditions, or other measurables, or otherwise monitoring a patient under observation. For example, portable monitoring devices 113, 114 may be configured as a pulse oximeter for measuring the percent oxygen saturation of hemoglobin in arterial blood (SpO₂). In particular, first and second sensors 115, 116 disposed on the patient's right hand and left foot, respectively, may be provided for sensing SpO₂ levels at these locations and relaying the same to portable monitoring device 114, although greater or fewer sensors and/or sensors in different locations may also be provided, depending on a particular purpose. Further, sensors 115, 116 may be connected to the same device (as shown), or each sensor 115, 116 may be connected to a different device. Portable monitoring device 114 may be configured to process and display the SpO₂ data (or other patient data) on a visual display 117 thereof and/or may be configured to relay the SpO₂ data (or other patient data) to one or more servers 120, 130, 140, e.g., data server 120, this may be done wirelessly as shown with respect to portable monitoring device 114, or as a wired connection as shown with respect to portable monitoring device 113. Bedside monitoring devices 111, 112 and portable monitoring devices 113, 114 may similarly be employed to monitor other characteristics, conditions, measurables, or to otherwise monitor the patient and to process the patient data, display the patient data, and/or relay the patient data to data server 120. Patient monitoring devices 110 may be wirelessly coupled to data server 120, or may be coupled to data server 120 via a wired connection. Patient monitoring devices 110 may include any suitable software, firmware, and hardware for these purposes.

Data server 120, as mentioned above, is configured to receive patient data from patient monitoring devices 110, although application server 130 and/or web server 140 may additionally or alternatively be configured to receive patient data from patient monitoring devices 110. One or more of servers 120, 130, 140 may further be configured to store the patient data in a database, process the patient data, and/or transmit the patient data between servers 120, 130, 140, to patient monitoring devices 110, and/or to remote devices 150. Servers 120, 130, 140 may include any suitable software, firmware, and hardware for these purposes.

Remote devices 150 request and receive the patient data, process the patient data, if needed, and display the patient data to a user, e.g., via a display monitor, user interface, browser, and/or application running on the remote device 150. Remote devices 150 may further be configured to receive input from a user, e.g., to manipulate the displayed data, set parameters for the displayed data, etc. Remote devices 150 may include any suitable software, firmware, and hardware for these purposes.

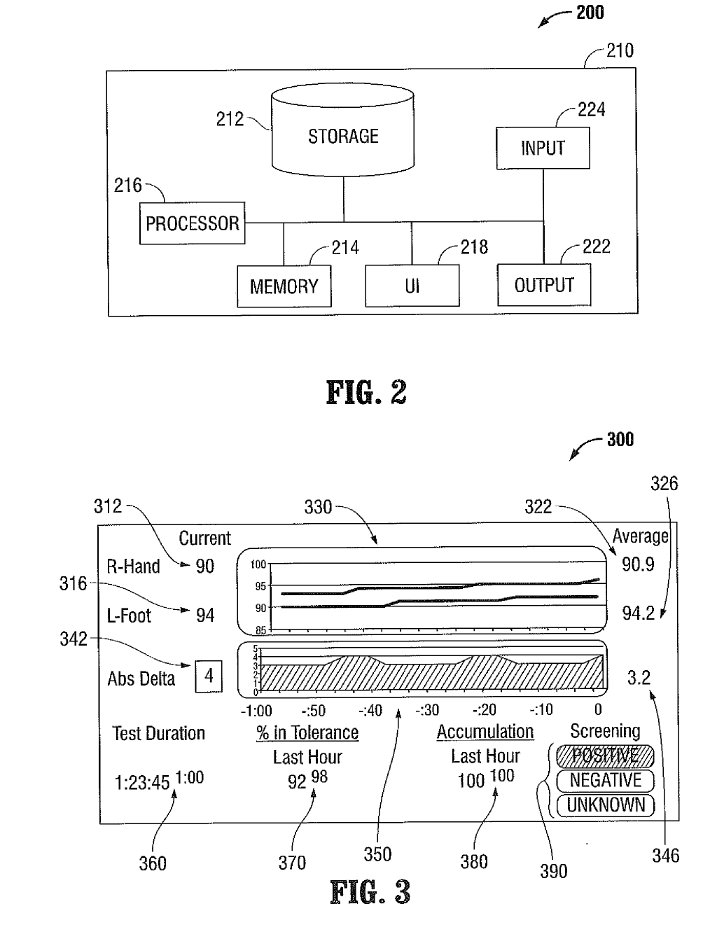

Turning now to FIG. 2, in conjunction with FIG. 1, one configuration of hardware and software components for receiving/transmitting patient data, processing patient data, receiving user input, and/or displaying patient data in accordance with the present disclosure is shown generally identified by reference numeral 200. Configuration 200 may be embodied within one or more of patient monitoring devices 110, servers 120, 130, 140, and/or remote devices 150, or may be implemented across one or more of patient monitoring devices 110, servers 120, 130, 140, and/or remote devices 150. That is, receiving/transmitting the patient data and user input, processing the patient data, and outputting the patient data for display may be performed locally, e.g., at one of patient monitoring devices 110, on one or more servers 120, 130, 140 for distribution to patient monitoring devices 110 or remote devices 150, e.g., across a network, at the remote devices 150 themselves, or in any combination of the above. For the purposes of simplicity, configuration 200 will be described herein as embodied in a system 210, keeping in mind that system 210 may be incorporated into any or all of the components of system 10.

System 210 generally includes a storage 212, a memory 214, a processor 216, a user interface (UI) 218, an output 222, and an input 224. Storage device 212 may include any suitable component(s) operable for storing data, e.g., patient data received via input 224, such as, for example, a magnetic disk, flash memory, optical disk, or other suitable data storage device. Memory 214 may include any computer memory, e.g., RAM or ROM, mass storage media, removable storage media, combinations thereof, or any other suitable computer-readable storage medium, storing instructions for causing processor 216 to execute particular functions, e.g., to process the patient data. Processor 216 may include any suitable component(s), e.g., a central processing unit (CPU), operable to execute instructions stored in memory 214 to process and manipulate patient data, e.g., stored in storage device 212 or received via input 224, for output to UI 218 or to output 222. Processor 216 is further configured to receive, via input 224 and/or US 218, information, data, and/or control parameters for processing and manipulating the patient data in accordance with user-selected settings and user input. UI 218 functions to output the processed patient data for visual display, e.g., in graphical and/or numerical form, to the user and/or allows for the input of data, setting of parameters, etc., by the user. Output and input 222, 224, respectively, are provided to facilitate communication between system 210 and the other components of system 10. In particular, input 224 is configured to receive patient data to be processed, e.g., data sensed by first and second sensors 115, 116.

Turning now to FIG. 3, in conjunction with FIGS. 1 and 2, a main display screen 300 is show displaying exemplary patient data as output by UI 218. Main display screen 300 may represent the visual display 117 of portable monitoring device 114 (or other patient monitoring device 110), or a monitor, display, etc. of one of remote devices 150. That is, depending on the configuration of system 10, the user may view display screen 300 via one or more of patient monitoring devices 110, remote devices 150, or other device. Main display screen 300 provides information and data to the user including numerical values 312, 316 representing the current SpO₂ readings at the respective first and second sensors 115, 116, e.g., right hand sensor 115 and left foot sensor 116, and the average SpO₂ values 322, 326 at the respective first and second sensors 115, 116 over a time period; a graphical representation 330 of the SpO₂ readings at the respective first and second sensors 115, 116 showing the change over the time period of the values both individually and relative to one another; a current differential value 342 representing the difference between the current SpO₂ readings at the respective first and second sensors 115, 116; an average differential value 346 representing the average difference between the SpO₂ readings at the respective first and second sensors 115, 116 over the time period; a graphical representation 350 of the differential value over the time period; an elapsed time clock 360; a “% in Tolerance,” or tolerance value 370; an accumulation value 380; and a screen result indicator 390, each of which will be described in greater detail below.

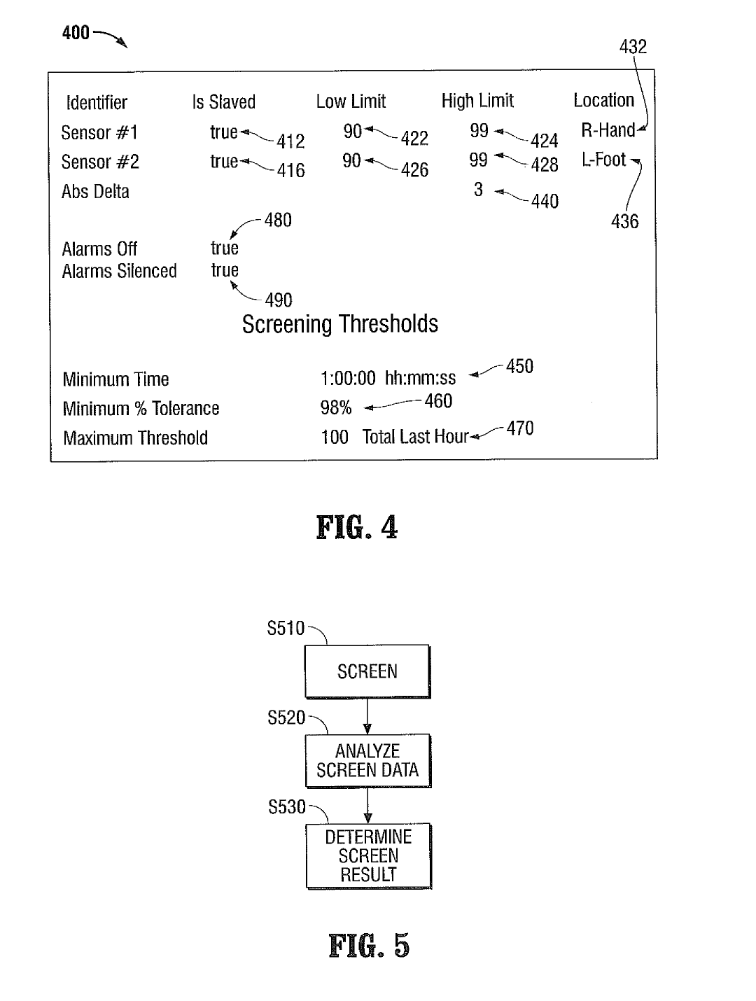

Referring to FIG. 4 in conjunction with FIGS. 1-3, a setting screen 400 is shown displaying exemplary setting information. Setting screen 400 may be accessed via the visual display 117 of portable monitoring device 114 (or other patient monitoring device 110), or the monitor, user interface, display, etc. of one of remote devices 150, depending on the configuration of system 10. By accessing setting screen 400, the user is able to input data and/or parameters, ultimately to be received by processor 216 for processing the patient data in accordance with user-selected settings. Alternatively or additionally, the settings populating setting screen 400 may be acquired automatically from other components of system 10 or may be default values in instances where the user has not selected a particular setting. Setting screen 400 is configured to display to the user, e.g., for parameter setting and/or confirmation of settings, identification indicators 412, 416 indicating whether the respective sensors 115, 116 are properly connected, high and low threshold values 422, 424 and 426, 428, respectively, for each sensor 115, 116, a location indicator 432, 436 indicating the location of the respective sensors 115, 116; a threshold 440 for the differential value; a minimum period of time 450 for the screening; a “Minimum % Tolerance” or minimum tolerance 460; a threshold 470 for the accumulation value; and alarm indicators 480, 490. Each of these which will be described in greater detail below.

With general reference to FIGS. 1-4, as mentioned above, first and second sensors 115, 116 may be configured to sense SpO₂ levels at various locations, e.g., at the right hand and left foot, and transmit corresponding signals for processing and displaying to a user. In accordance with the present disclosure, the SpO₂ data from first and second sensors 115, 116 is used to calculate various indicators or metrics and/or may be otherwise analyzed to provide the user with a screen result, indicating whether the patient's SpO₂ levels are within acceptable limits (negative screen result), outside of acceptable limits (positive screen result), or that the result is unknown, e.g., due to technical error or inconclusive data. Alternatively or additionally, the data, indicators, and/or metrics may be displayed in various forms to the user to assist in determining a screen result or monitoring the patient. Although the processes and methods are described below for exemplary purposes with respect to screening patient, e.g., screening newborns for CCHDs, by monitoring SpO₂ levels at two different locations, the present disclosure is not limited to screening, as it is contemplated that the aspects and features of the present disclosure be applicable for use in the monitoring of SpO₂ levels for any suitable purpose. Obviously, the settings, parameters, and thresholds may change depending on a particular purpose; however, the general features and aspects of the present disclosure remain generally consistent regardless of the particular purpose. Further, the features and aspects of the present disclosure may be implemented in system 10 in any suitable fashion, e.g., via the hardware and software configuration 200 of system 210 or using any other suitable software, firmware, and/or hardware.

Referring to FIG. 4, in conjunction with FIGS. 1-3, prior to screening, the user may input the desired settings corresponding to the particular patient to be screened, the purpose of the screen, and/or other factors via accessing setting screen 400. In the absence of user-input settings, default settings may be used. With respect to the settings, the user may confirm proper connection of the first and second sensors, as shown by the “true” indicators 412, 416, respectively, under the “Is Slaved” column of setting screen 400. The user may also select the location of each of the sensors, “R-Hand” and “L-Foot,” for example, as indicated under the “Location” column by indicators 432 and 436, respectively, although the sensor locations may alternatively be identified automatically. “Low Limit” and “High Limit” columns allow for the user to set and confirm the respective limits for each sensor, as seen by indicators 422, 424 and 426, 428, respectively. As will be described below, the low limit for each sensor may correspond to the respective threshold values used in determining the screen result, while the high limits may serve as an error-check to ensure the sensors are operating properly (because, for example, a proper SpO₂ reading cannot exceed 100, as SpO₂ is a percentage). The user may also set and confirm the maximum differential threshold, as provided by indicator 440. Low and high limits and differential thresholds may automatically be populated as default values depending on the location of the sensor selected. For example, particular limits and thresholds may be desirable for configuration wherein the first sensor 115 is disposed on the right hand of the patient and the second sensor 116 is disposed on the left foot of the patient.

With continued reference to FIG. 4, in conjunction with FIGS. 1-3, alarm settings may also be set and/or confirmed via setting screen 400, e.g., turning alarms ON/OFF, as provided by indicator 480, and/or silencing alarms, as provided by indicator 490. The alarms may be set to ON/OFF or silenced as a group (as shown) or individually (not shown). The alarms may include high-limit alarms, low-limit alarms, differential threshold alarms, sensor disconnected alarms, or any other suitable alarm configured to alert the user (audibly and/or visually) as to a particular condition during the screen. The alarms may be output to any of patient monitoring devices 110 and/or remote devices 150.

The “Screening Threshold” settings of minimum time 450, minimum tolerance 460 and maximum accumulation threshold 470 allow the user to further customize the screening for a particular purpose. The minimum time corresponds to the minimum screening time. That is, a result determination will not be registered unless the minimum screening time has elapsed. Such a feature helps ensure that a sufficient amount of data, over a sufficient amount of time, is obtained so as to promote accuracy and minimize the bias of data anomalies. The maximum accumulation threshold and minimum tolerance will be described in greater detail below.

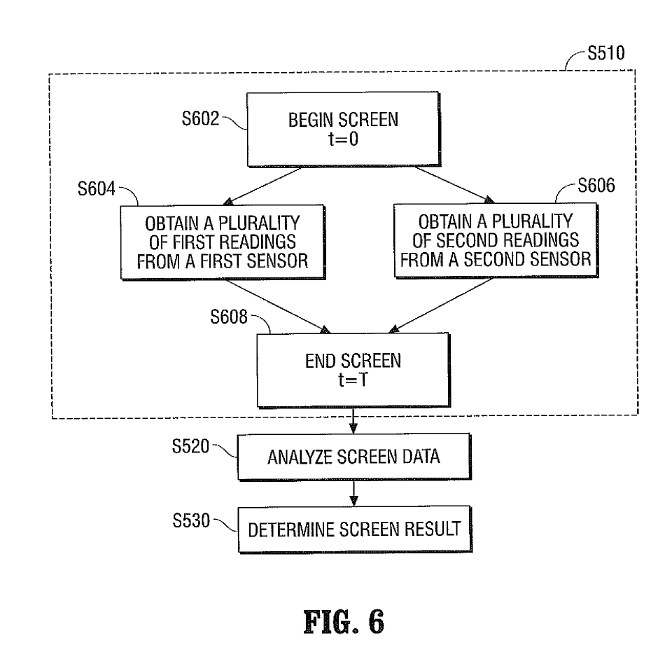

Referring to FIG. 5, in conjunction with FIGS. 1-4, SpO₂ screening is generally accomplished via screening the patient (S510), analyzing the screen data (S520), and determining the screen result based on the analysis of the screen data (S530). Each of these steps will be described in greater detail with reference to FIG. 6-10, in conjunction with FIGS. 1-4. For the purposes herein, “differential value” is calculated as, and refers to, an absolute value. Further, as utilized herein, a “positive” screen result corresponds to a failed screen, wherein one or more of the patient's SpO₂ levels and/or the corresponding values calculated therefrom are outside their respective acceptable ranges, thus indicating that the patient may potentially be unhealthy or at-risk. A “negative” screen result, on the other hand, corresponds to a passed screen, indicating that the patient's SpO₂ levels and/or the corresponding values calculated therefrom are within their acceptable ranges.

Turning to FIG. 6, in conjunction with FIGS. 1-4, with respect to screening the patient (S510), sensors 115, 116 are each utilized to obtain a plurality of SpO₂ readings over a predetermined length of time. More specifically, screening beings at time t=0 (S602) and ends at time t=T (S608). Time T may be set by the user by accessing setting screen 400 and, more particularly, by setting a desired minimum period of time 450 for the screening. The selected (or default) minimum time T is displayed to the user on the main display screen 300 as a superscript of the elapsed time 360. As mentioned above, a screen result will not be determined until the minimum time T has elapsed, although screening continues even after time T has elapsed. If screening is ended before the minimum time T has elapsed, an unknown screen result will be indicated.

During screening (S510), as mentioned above, a plurality of first readings are obtained by first sensor 115 and a plurality of second readings are obtained by second sensor 116 (S604 and S606, respectively) over time t=0 to t=T. The intervals at which readings are taken may be constant, e.g., one reading every second, and/or may be set by the user to provide a desired level of granularity. In either configuration, the readings may be taken manually by a user or automatically taken by sensors 115, 116 and/or portable patient monitoring devices, 113, 114, e.g., independently of the user, at the prescribed intervals. The readings, obtained from sensors 115, 116 in the form of electrical signals, are input into input 224 of system 210, which may be partially or wholly embodied within portable patient monitoring device 114, one or more of the other patient monitoring devices 110, one or more of servers 120, 130, 140, and/or one or more of remote devices 150. The electrical signals received from sensors 115, 116 are converted into SpO₂ readings, e.g., via processor 216, and are stored in a database, e.g., in storage 212. An exemplary data set of SpO₂ readings obtained from first and second sensors 115, 116, respectively, is provided in TABLE 1:

| Interval | First Sensor SpO2 | Second Sensor SpO2 |
|---|---|---|
| (Time) | Reading | Reading |
| 0 (t = 0) | 94 | 92 |
| 1 | 94 | 91 |
| 2 | 94 | 90 |
| 3 | 94 | 90 |
| 4 | 94 | 90 |
| 5 | 94 | 90 |
| 6 | 82 | 90 |
| 7 | 84 | 90 |
| 8 | 80 | 85 |
| 9 | 94 | 92 |
| 10 | 94 | 92 |
| 11 | 94 | 92 |
| 12 | 94 | 92 |
| 13 | 94 | 93 |
| 14 | 94 | 93 |
| 15 | 94 | 94 |
| 16 (t = T) | 94 | 94 |

During screening, as mentioned above, the current SpO₂ readings are displayed on main display screen 300 numerically as indicated by reference numerals 312, 316. A graphical representation 330 of past readings from time t=0 (or a previous time period) to current is also displayed on main display screen 300. Once screening is complete, the screen data is analyzed (S520) to determine a screen result (S530), as will be described in greater detail below.

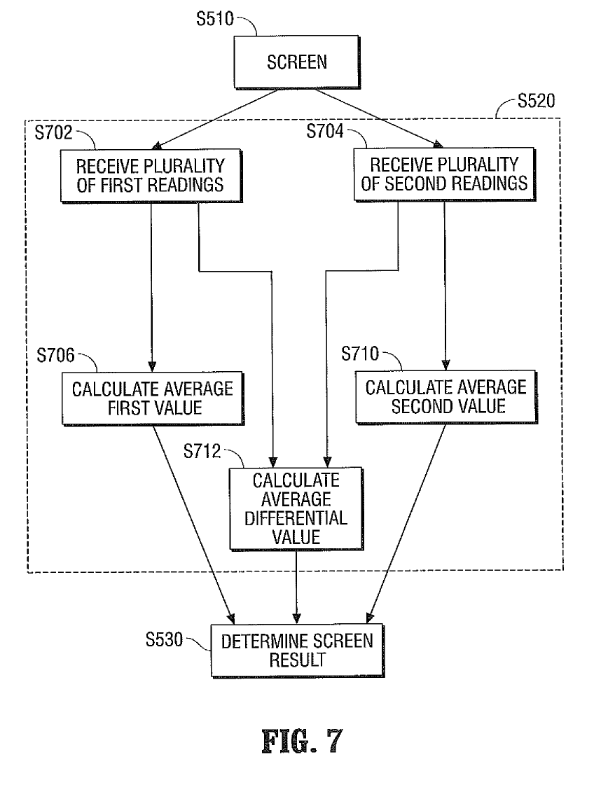

Turning now to FIG. 7, in conjunction with FIGS. 1-4, analyzing the screen data (S520) may be performed by processor 216 and may include receiving the first and second plurality of readings (S702, S704, respectively) from sensors 115, 116, storage 212, or input 224 (via one of the patient monitoring devices 110, servers 120, 130, 140, or remote devices 150), and calculating an average first value (S706) corresponding to an average of the plurality of first readings, an average second value (S710) corresponding to an average of the plurality of second readings, and an average differential value (S710) corresponding to the average differential value between the first reading and second reading at each interval. As mentioned above, all differential values are provided as absolute values. Calculating the average differential value S710 may initially include calculating a plurality of differential values corresponding to the differential value at each interval, from which the average differential value is be calculated. These calculated values may likewise be stored in storage 212. The current differential value, updated after each interval, is displayed on main display screen 300 as indicated by reference numeral 342. A graphical representation 350 of past differential values from time t=0 (or a previous time period) to current is also displayed on main display screen 300. The plurality of differential values calculated from the exemplary data set in TABLE 1, above, and used in calculating the average differential value are provided in TABLE 2:

| Interval (Time) | Differential Value |
|---|---|
| 0 (t = 0) | 2 |
| 1 | 3 |
| 2 | 4 |
| 3 | 4 |
| 4 | 4 |
| 5 | 4 |
| 6 | 8 |
| 7 | 6 |
| 8 | 5 |
| 9 | 2 |
| 10 | 2 |
| 11 | 2 |
| 12 | 2 |
| 13 | 1 |
| 14 | 1 |
| 15 | 0 |
| 16 (t = T) | 0 |

The average values of the plurality of first and second readings and the average differential value are calculated cumulatively and continuously, e.g., the averages are recalculated after each interval to include the readings corresponding to the next successive interval, and are displayed on main display screen 300 as indicated by reference numerals 322, 326, and 346, respectively. The average first and second values and the average differential value calculated from the exemplary data set of SpO₂ values in TABLE 1, above, is provided in TABLE 3:

| Average First Value | Average Second Value | Average Differential Value |
|---|---|---|
| 91.9 | 91.2 | 2.9 |

Based on the average first value, average second value, and average differential value, a screen result (S530) can be determined, as will be described in greater detail below. Alternatively, where only monitoring a patient is desired (as opposed to screening a patient to determine a particular result), the graphical and numerical displays of the current and average first values, current and average second values, and current and average differential values provide the user with an indication of the patient's SpO₂ levels at any particular point-in-time as well as over an elapsed period of time.

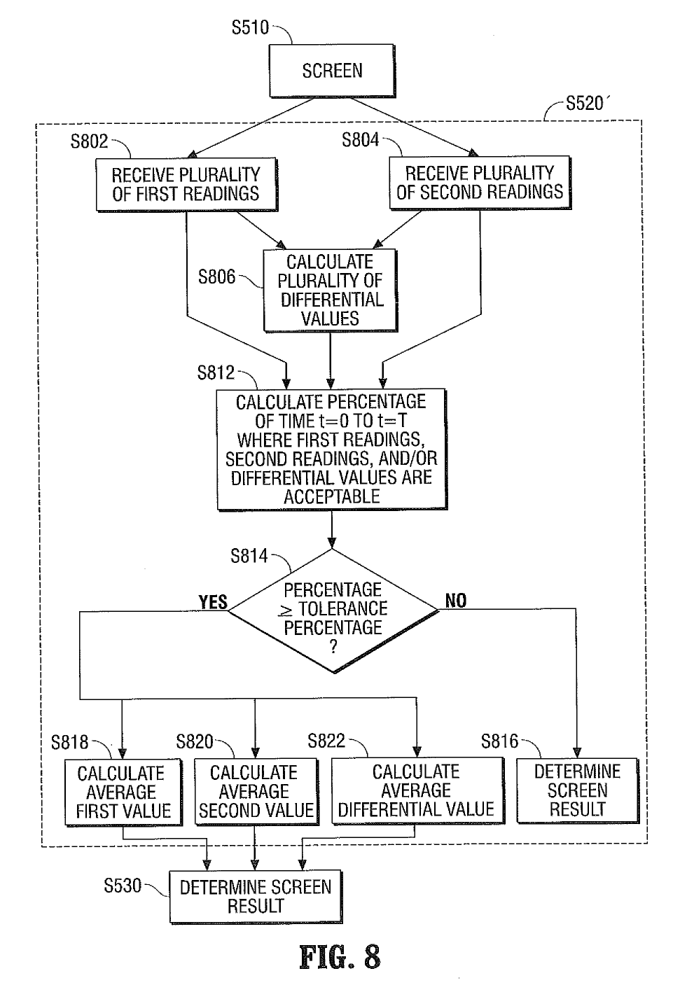

Turning now to FIG. 8, in conjunction with FIGS. 1-4, another implementation of analyzing the screen data (S520′) to be used additionally or alternatively, includes, similarly as above, receiving a plurality of first readings (S802) and receiving a plurality of second readings (S804). From these readings, a plurality of differential values (S806) corresponding to the difference between the first reading and second reading at each particular interval are calculated (and/or an accumulation differential value may be calculated, as described above). Analyzing the screen data (S520′) further includes calculating a percentage of time where the first readings, second readings, and/or differential values are acceptable (hereinafter “tolerance value”) (S812), e.g., within a tolerable range. More specifically, a percentage of time (from t=0 to t=T) wherein the first reading is greater or equal to a first threshold, the second reading is greater or equal to a second threshold, and/or the differential value is less than or equal to a differential threshold value is calculated. The acceptable thresholds, or limits 422, 426, 440 for each of the first readings, second readings, and differential values, respectively, may be set or confirmed by accessing the setting screen 400, as detailed above. The tolerance value is calculated cumulatively and continuously, e.g., the tolerance value is recalculated after each interval, and is displayed on main display screen 300, as indicated by reference numeral 370. As an example, using the exemplary data of TABLE 2, above, and a threshold differential value of 4, the percentage of time the differential value is less than or equal to the differential threshold value is approximately 82% (because the differential value is equal to or less than 4 in 14 of the 17 interval readings). This tolerance value represents the percentage of time (from t=0 to t=T) wherein the differential value between each first reading and the corresponding second reading is within an acceptable or tolerable range. However, as mentioned above, the tolerance value may be additionally or alternatively be calculated as the percentage of time when the first readings are greater or equal to the first threshold, the second readings are greater or equal to the second threshold, or combinations thereof. Further, multiple tolerance values may be calculated, each corresponding to a different value or group of values. In such embodiments, the below-described process may be repeated for each tolerance value.

Once calculated, the tolerance value is compared to a minimum tolerance (S814), e.g., a minimum percentage of time wherein the readings and/or values are acceptable, which may be set or confirmed via the indicator 460, “Minimum % Tolerance,” of setting screen 400. The tolerance value, as indicated by reference numeral 370, is displayed on main display screen 300, while the minimum tolerance is displayed on main display screen 300 as a superscript of the percentage in tolerance value. If the tolerance value is less than the minimum tolerance (“NO” in S814), the process proceeds to S816, wherein the screen result can be determined without further analysis/determination, e.g., solely on the tolerance value (or values, where multiple tolerance values are provided). The determined result may depend on the particular data used in calculating the tolerance value, e.g., whether the first readings, second readings, and/or differential values are used. For example, where the first and/or second readings are used in determining the tolerance value, it can be determined that the screen result is positive if the tolerance value is less than the minimum tolerance, as the tolerance value indicates that a substantial percentage of the first and/or second readings were outside their respective limits. As another example, where only the differential values are used, the result may be an unknown test result, because the tolerance value indicates that a significant portion of the data obtained over the time period t=0 to t=T may be unreliable. Alternatively or additionally, the tolerance value (whether or not it exceeds the minimum tolerance) may be provided as one of several metrics displayed on main display screen 300 from which the user can determine the screen result or use in evaluating the screen data.

With momentary reference to FIG. 3, accumulation, which is displayed on main display screen 300 via numerical indicator 380, is another metric which may be used in determining the screen result or in evaluating the screen data, alone or in combination with the average differential value, tolerance value, and/or other data and metrics. Accumulation corresponds to the area under the differential value curve over the screening time period (t=0 to t=T), e.g., the accumulated differential value between the plurality of first and second readings, which is displayed on main display screen 300 as graphical representation 350. In utilizing accumulation to determine the screen result, the accumulation value, i.e., the accumulated differential value, is compared to the accumulation value threshold 470 (FIG. 4), which may be set or confirmed via the indicator 460, “Maximum Threshold,” of setting screen 400 (FIG. 4). Comparison of the accumulation value to the accumulation value threshold 470 (FIG. 4) may be used in place of, or in conjunction with, the average differential value and corresponding third threshold in determining the screen result, as will be described in greater detail below. Alternatively, accumulation may correspond to the area under either or both of the first and second readings curves (displayed on main display screen 300 as graphical representation 350) relative to the respective threshold values thereof, similar to the SatSeconds™ Alarm Management Technology feature described in U.S. Pat. No. 5,865,736, the entire contents of which are hereby incorporated by reference herein.

Referring again to FIG. 8, in conjunction with FIGS. 1-4, if the tolerance value is greater than the minimum tolerance (“YES” in S814), the process proceeds to steps S818, S820, and S822, wherein, similarly as above, the average first value, average second value, and average differential value are calculated for ultimately determining the screen result (5530), as will be described in greater detail below. Alternatively, even where the tolerance value exceeds the minimum tolerance (“YES” in S814), a screen result determination can be made without further calculation, depending on the data used for the tolerance value calculation. For example, where the first readings, second readings, and differential values are all used in determining the tolerance value, it can be determined that the screen result is negative if the tolerance value exceeds the minimum tolerance, as the tolerance value would thus indicate that a substantial percentage of each of these values are within their respective limits. On the other hand, where only differential value is used, for example, the fact that the tolerance value exceeds the minimum tolerance may indicate that the data obtained over the time period t 0 to t=T is reliable data from which a result can ultimately be determined (S530).

In general, the tolerance value provides an indication as to the test result and/or test reliability over the time t=0 to t=T. That is, a tolerance value below the minimum tolerance may indicate a significant number of positive readings or a significant number of unreliable readings during screening, despite the fact that the readings at any given point-in-time may be within acceptable ranges. This feature helps prevent false negative results. On the other hand, a tolerance value above the minimum tolerance may indicate a large majority of negative readings or at least that there is no sensor error, despite the fact that infrequent and/or minimally significant spikes may occur at any given point-in-time to help prevent false positive or false unknown results. As will be described below, these spikes may be caused by patient movement and may not accurately reflect actual SpO₂ levels. The present disclosure contemplates eliminating the data corresponding to these spikes from use in the screen result determination to further help prevent false positive results.

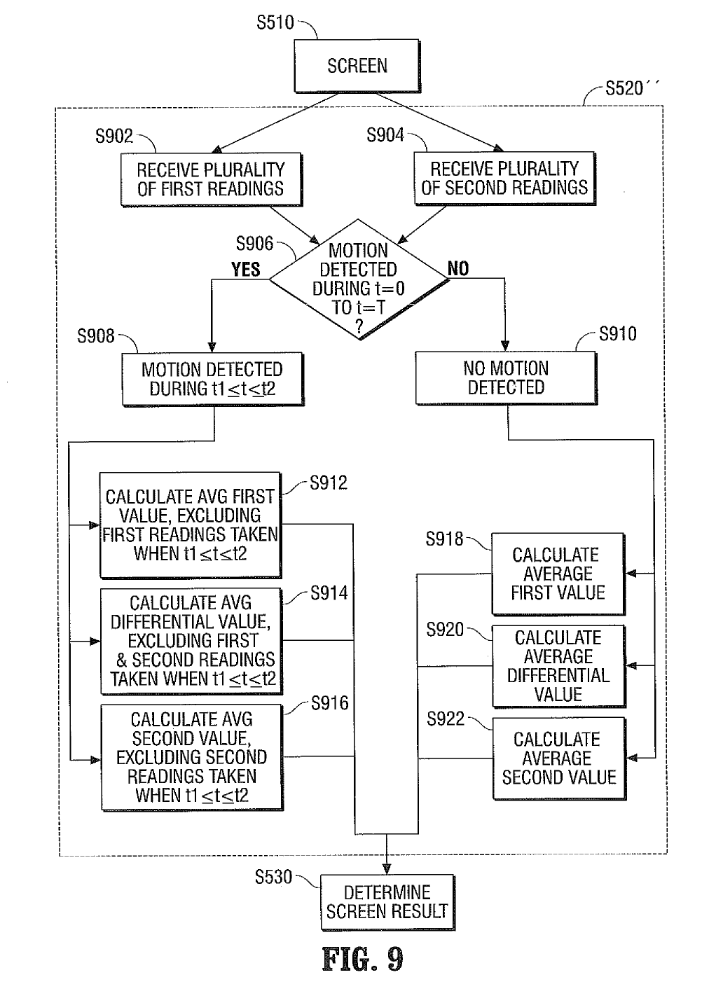

Turning now to FIG. 9, in conjunction with FIGS. 1-4, another implementation of analyzing the screen data (S520″) to be used as an alternative or in addition to the other implementations described above includes receiving a plurality of first readings (S902) and receiving a plurality of second readings (S904) over the course of time t=0 to t=T. Analyzing the screen data (S520″) further includes determining whether and when patient movement, or motion, has occurred during t=0 to t=T (S906). Motion may be sensed via any suitable sensor (not shown), e.g., a motion sensor, other sensor configured to detect indicators of motion, a video camera, etc., that may be incorporated into one of patient monitoring devices 110. Alternatively, motion may be inferred from the plurality of first and second readings. Because SpO₂ readings may be significantly affected by patient movement, motion sensing and/or SpO₂ readings that are significantly off-base may be used as a proxy for discarding particular data from use in determining the screen result. Motion sensing may include providing a suitable sensor (not shown) capable of determining a time period (or time periods) during time t=0 to t=T wherein motion (or motion above a threshold level of motion) occurs, and relaying this information to processor 216. With respect to inferring motion from the plurality of first and second readings, processor 216 may be configured to determine a time period (or time periods) during time t=0 to t=T wherein the first and/or second plurality of readings are outside a range which would clearly indicate motion. The particular range may be a range stored in storage 212, or a range calculated based on deviation from nearby-in-time readings, e.g., using readings before and after the time period in question. If motion (or significant motion about a threshold) is detected (“YES” in S906), the process proceeds to step S908, as will be described in greater detail below. On the other hand, if no motion is detected (or insignificant motion is detected) (“NO” in S906), the process proceeds to step S910, wherein, similar to above, the average first value (S918), average second value (S920), and average differential value (S920) are calculated, from which the screen result can be determined (S530), as will be described in greater detail below.

With regard to motion being detected (S908), for example, during the time period t1 to t2, any readings from the time period t1 to t2 are excluded from the calculation of the average values and average differential value (S912, S915, S916, respectively). For example, using the exemplary data of TABLE 1, above, if motion is detected from time intervals 6 through 8, these values would be eliminated from calculating the average values and average differential value (S912, S915, S916, respectively). The average values and average differential values in this example, wherein the readings from intervals 6 through 8 are excluded, are shown in TABLE 4:

| Average First Value | Average Second Value | Average Differential Value |
|---|---|---|
| 94.0 | 91.8 | 2.2 |

As can be appreciated, and as shown via comparison of TABLES 3 and 4, eliminating readings taken during periods of motion serves to provide a more accurate indication of the patient's true SpO₂ levels. It is further contemplated that other known conditions that may alter screening data may be sensed and those time periods also eliminated from calculating the above-described values, e.g., periods of technical error (sensor malfunction, sensor disconnection, weak signals), periods of other physical or physiological conditions (sleeping, coughing), etc.

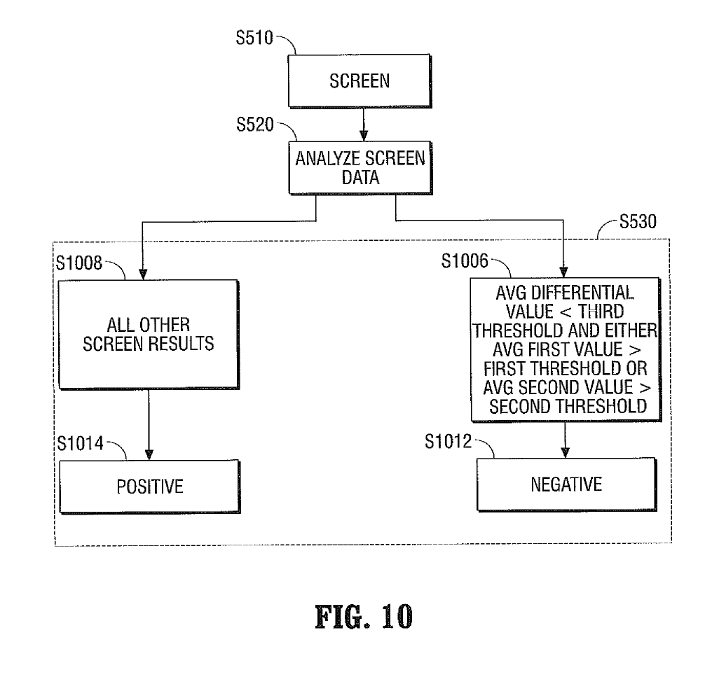

Turning now to FIG. 10, in conjunction with FIGS. 1-4, using the average first value, the average second value, and average differential value, a screen result may be determined, as indicated in step S530. More specifically, as indicated in S1006, if the average differential value is less than the third threshold and either the average first value is greater than the first threshold or the average second value is greater than the second threshold, the screen result is determined to be “NEGATIVE” (S1012) because the average differential value is within acceptable limits and at least one of the first and second average values is above its respective threshold. For all other screen results (S1008), e.g., screen results that not meet the above criteria, the screen result is determined to be “POSITIVE” (S1014).

As mentioned above, accumulation may be used to determine the screen result. More specifically, as an alternative or in addition to determining whether the average differential value is less than the third threshold, the accumulation value, i.e., the accumulated differential value, may be compared to the accumulation value threshold to determine whether the accumulation value is less than the accumulation value threshold. If the accumulation value is less than the accumulation value threshold (and/or the average differential value is less than the third threshold) and either the average first value is greater than the first threshold or the average second value is greater than the second threshold, the screen result is determined to be “NEGATIVE” because the accumulation value is within acceptable limits and at least one of the first and second average values is above its respective threshold. Otherwise, the screen result is determined to be “POSITIVE.”

The above-described screen result determination in step S530 may be used as the sole determining factor, from which processor 216 may signal UI 218 and/or output 222 to display a corresponding result on main display screen 300 via the screen result indicator 390. Alternatively, the screen result determination in step S530 may be used in conjunction with any or all of the other above-described data, metrics, and calculations in determining a result, depending on a particular purpose. For example, processor 216 may be configured to signal UI 218 and/or output 222 to display a corresponding screen result based upon the determine screen result step S530 and for example, the tolerance determination. That is, a screen result may be displayed as “POSITIVE” or “NEGATIVE” only if the both the determine screen result step S530 and the tolerance determination (detailed above) provide a similar result, while an “UNKNOWN” result is displayed otherwise. Alternatively, all of the above-described data, metrics, and calculations, may be presented to a user, e.g., via main display screen 300, such that the user may make a determination as to the screen results based on this information and depending on the particular patient, purpose of the screen, or other factors.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A method of screening a patient, comprising the steps of:
receiving a plurality of first SpO₂ readings from a first sensor over a time period;
receiving a plurality of second SpO₂ readings from a second sensor over the time period;
calculating an average first SpO₂ value based on at least some of the plurality of first SpO₂ readings;
calculating an average second SpO₂ value based on at least some of the plurality of second SpO₂ readings;
calculating a differential value based on at least some of the plurality of first SpO₂ readings and at least some of the plurality of second SpO₂ readings; and
determining a screen result based upon the average first SpO₂ value, the average second SpO₂ value, and the differential value.

2. The method according to claim 1, wherein the differential value is an average differential value, and wherein the method further includes the step of calculating a plurality of differential values, each differential value calculated as an absolute value difference between one of the plurality of first SpO₂ readings and a corresponding one of the plurality of second SpO₂ readings, wherein the average differential value is calculated as an average of at least some of the plurality of differential values.

3. The method according to claim 1, wherein the differential value is an accumulation differential value, and wherein the method further includes the step of calculating a plurality of differential values, each differential value calculated as an absolute value difference between one of the plurality of first SpO₂ readings and a corresponding one of the plurality of second SpO₂ readings, wherein the accumulation differential value is calculated as a sum of at least some of the plurality of differential values.

4. The method according to claim 1, wherein the screen result is determined to be negative if the differential value is less than a third threshold and either the average first SpO₂ value is greater than a first threshold or the average second SpO₂ value is greater than a second threshold.

5. The method according to claim 1, wherein the screen result is determined to be unknown if the time period is less than a minimum time threshold.

6. The method according to claim 1, wherein all of the first SpO₂ readings within a selected portion of the time period and all of the second SpO₂ reading within the selected portion of the time period are excluded from use in calculating the average first SpO₂ value, the average second SpO₂ value, and the differential value.

7. The method according to claim 1, further comprising the step of determining a percentage of the time period when at least one of the first SpO₂ values are greater than a first threshold, the second SpO₂ values are greater than a second threshold, and the differential values between the first SpO₂ readings and the second SpO₂ readings are less than a third threshold.

8. A system for screening a patient, comprising:
a first sensor disposed at a first location, the first sensor configured to obtain a plurality of first SpO₂ readings over a time period;
a second sensor disposed at a second location, the second sensor configured to obtain a plurality of second SpO₂ readings over the time period;
a processor in operable communication with the first and second sensors, the processor configured to:
receive the plurality of first SpO₂ readings from the first sensor;
receive the plurality of second SpO₂ readings from the second sensor;
calculate an average first SpO₂ value based on at least some of the plurality of first SpO₂ readings;
calculate an average second SpO₂ value based on at least some of the plurality of second SpO₂ readings;
calculate a plurality of differential values, each differential value calculated as a difference between one of the plurality of first SpO₂ readings and a corresponding one of the plurality of second SpO₂ readings; and
calculate at least one of an average differential value and an accumulation differential value based on at least some of the plurality of differential values; and
a display in operable communication with the processor, the display configured to:
display a current first SpO₂ reading;
display the average first SpO₂ value;
display a current second SpO₂ reading;
display the average second SpO₂ value;
display a current differential value; and
display the at least one of the average differential value and the accumulation differential value.

9. The system according to claim 8, wherein the processor is further configured to determine a screen result based upon the average first SpO₂ value, the average second SpO₂ value, and the at least one of the average differential value and the accumulation differential value, and wherein the display is further configured to display the screen result.

10. The system according to claim 9, wherein the processor determines the screen result to be negative if the at least one of the average differential value and the accumulation differential value is less than or equal to a respective threshold and either the average first SpO₂ value is greater than a first threshold or the average second SpO₂ value is greater than a second threshold.

11. The system according to claim 8, wherein the average differential value is calculated as an average of at least some of the plurality of differential values.

12. The system according to claim 8, wherein the accumulation differential value is calculated as a sum of at least some of the plurality of differential values.

13. The system according to claim 8, wherein the processor is further configured to exclude all of the first SpO₂ readings within a selected portion of the time period and all of the second SpO₂ reading within the selected portion of the time period from use in calculating the average first SpO₂ value, the average second SpO₂ value, and the at least one of the average differential value and the accumulation differential value.

14. The system according to claim 8, wherein the processor is further configured to determine a percentage of the time period when at least one of the first SpO₂ values are greater than or equal to a first threshold, the second SpO₂ values are greater than or equal to a second threshold, and the differential values between the first SpO₂ readings and the second SpO₂ readings are less than or equal to a third threshold, and wherein the display is further configured to display the percentage of the time period.

15. A non-transitory computer-readable storage medium encoded with a program that, when executed by a processor, causes the processor to:
receive a plurality of first SpO₂ readings from a first sensor over a time period;
receive a plurality of second SpO₂ readings from a second sensor over the time period;
calculate an average first SpO₂ value based on at least some of the plurality of first SpO₂ readings;
calculate an average second SpO₂ value based on at least some of the plurality of second SpO₂ readings;
calculate a plurality of differential values, each differential value calculated as a difference between one of the plurality of first SpO₂ readings and a corresponding one of the plurality of second SpO₂ readings;
calculate at least one of an average differential value and an accumulation differential value based on at least some of the plurality of differential values; and
determine a screen result based upon the average first SpO₂ value, the average second SpO₂ value, and the at least one of the average differential value and the accumulation differential value.

16. The non-transitory computer-readable storage medium according to claim 15, wherein the processor is further caused to exclude all of the first SpO₂ readings within a selected portion of the time period and all of the second SpO₂ reading within the selected portion of the time period time from use in calculating the average first SpO₂ value, the average second SpO₂ value, and the at least one of the average differential value and the accumulation differential value.

17. The non-transitory computer-readable storage medium according to claim 15, wherein the processor is further caused to determine a percentage of the time period when at least one of the first SpO₂ values are greater than or equal to a first threshold, the second SpO₂ values are greater than or equal to a second threshold, and the differential values between the first SpO₂ readings and the second SpO₂ readings are less than or equal to a third threshold.

18. The non-transitory computer-readable storage medium according to claim 15, wherein the screen result is determined to be negative if the at least one of the average differential value and the accumulation differential value is less than a respective threshold and either the average first SpO₂ value is greater than a first threshold or the average second SpO₂ value is greater than a second threshold.

19. The non-transitory computer-readable storage medium according to claim 15, wherein the average differential value is calculated as an average of at least some of the plurality of differential values.

20. The non-transitory computer-readable storage medium according to claim 15, wherein the accumulation differential value is calculated as a sum of at least some of the plurality of differential values.
